(19) United States
(12) Patent Application Publication
(10) Pub. No.: US 2023/0408519 A1
(43) Pub. Date: Dec. 21, 2023

(54) Method and Apparatus for Detecting Bacterial, Viral and/or Parasitic Diseases

(71) Applicant: RoboScientific Ltd., Littleport (GB)

(72) Inventor: Stanley Curtis Mervyn, Soham (GB)

(21) Appl. No.: 18/251,423

(22) PCT Filed: Nov. 11, 2021
(86) PCT No.: PCT/GB2021/000121

§ 371 Date: May 2, 2023

(30) Foreign Application Priority Data
Nov. 11, 2020 (GB) 2017788.7
Nov. 16, 2020 (GB) 2018013.9
Nov. 16, 2020 (GB) 2018014.7
Jun. 10, 2021 (GB) 2018280.5

(51) Int. Cl. G01N 33/569; B01L 3/00; B01L 7/00

(52) U.S. Cl. CPC G01N 33/56983; B01L 3/5023; B01L 7/00; G01N 33/56911; B01L 2300/069; B01L 2300/1805; B01L 2300/0663

(57) ABSTRACT

Apparatus for detecting the presence in an enclosed environment of a subject or subjects infected with viral, bacterial and/or parasitic disease or diseases, the apparatus comprising: (a) an air sampling unit (1) able to take an air sample of the atmosphere in the enclosed environment and to divert said sample for sensing; (b) a selected definitive sensor set (9) comprising at least two sensors reactive to the presence of specific odours or Volatile Organic Compounds (VOCs) in the air sample taken from the environment; (c) a processing unit (10) comprising a pattern recognition analyser, wherein the pattern recognition analyser receives output signals of the sensor set, compares them to disease—specific patterns derived from a database of response patterns of the sensor set exposed to the totality of the bodily emissions of subjects with known disease or diseases, wherein each of the disease-specific patterns is characteristic of a particular disease, selected from bacteriological, viral and parasitic disease, and selects a closest match between the output signals of the sensor set and the disease-specific pattern; and (d) a control system that triggers the sampling of the air space of the environment at pre-determined times or intervals for rendering the apparatus entirely automatic and self-contained in operation, wherein the air sampling unit comprises: a surface (2) for capturing VOCs from the air sample; and a heater (3) for heating the surface to release captured VOCs when diverting the air sample for sensing.

### FIELD OF THE INVENTION

The present invention relates to a method and apparatus for detecting bacterial, viral and/or parasitic (e.g. malaria) diseases

### BACKGROUND ART

There is a requirement for a sensor system to detect when the carriers or victims of a disease are present or have been present in a particular location. When human or animal (e.g. poultry, cattle or pigs) subjects are infected with a disease they can cause cross infection through direct contact or by allowing droplets from breath; sweat or other bodily fluids to be suspended in the immediate atmosphere or to collect on surfaces or objects such as door handles or drink or food utensils. Thus one subject can infect one or more other subjects.

When large numbers of subjects (be they humans or animals) include one or more subjects who are infected with or carrying a disease it can be impractical, expensive and time consuming to test each subject individually. Existing test methods, such as LAMP and PCR tests, are invasive requiring typically a swab taken from the gums or from the nasal area and usually are undertaken by trained personnel. There is a need for a system which can sample the atmosphere of a known enclosed environment, such as a room, barn or poultry shed, and determine if any infected or disease carrying subjects are present or if any have recently been in the enclosed environment. Such a system would permit the pre-screening of large numbers of subjects for disease. As an example a building could have a multiple number of rooms. With the system monitoring each room outbreaks of disease could be quickly identified and the subjects of the specific rooms identified for individual testing. Furthermore using a tracking and tracing procedure recent occupants of those rooms could also be tested. Another example could be where a large number of aircraft are landing at an airport. The costs and time delays resulting from testing every passenger could render the whole process impractical. Alternatively if each aircraft was pre-screened by mounting the system or apparatus in the aircraft cabin then such aircraft containing infected subjects could be identified for individual passenger testing.

There are many methods of testing a subject for infection by a particular disease. These are largely based upon PCR or Elisa type of technology which are invasive requiring typically a swab taken from the gums or from the nasal area and usually they are undertaken by trained personnel. Such tests are expensive for large-scale testing particularly when conducted regularly. Thus there is a need for a testing method that is inexpensive to use; is non-invasive and less intrusive and which can give a result in seconds. Such a system would permit the pre-screening of large numbers of subjects for disease, allowing rapid interventions or further targeted testing and diagnosis.

The present invention also relates to a non-invasive sampling, sensing and detector system which will detect viral, bacterial and/or parasitic diseases causing illness in human subjects, by measuring and analysing and making comparison with the digital fingerprints of mixtures of odours or Volatile Organic Compounds (VOCs) in the breath or body sweat of the subject.

### SUMMARY OF THE INVENTION

The present invention in one aspect relates to a fully automatically operating and self-contained non-invasive sampling, sensing and detector system which will detect viral, bacterial and/or parasitic diseases causing illness in animals and human beings, by measuring and analysing and making comparison with the digital fingerprints of mixtures of odours or Volatile Organic Compounds in the environment atmosphere. The technique of detecting diseases by measuring and analysing and making comparison with the digital fingerprints of mixtures of odours or Volatile Organic Compounds is well established in the prior art with several inventions using breath samples from subjects.

Examples of this prior art include Neel (1994) U.S. Pat. No. 5,356,594 and Satterfield (2014) U.S. Pat. Application US 2014/0276100 A1.

The present invention is not based upon the use of a breath sample as the source of odours or Volatile Organic Compounds. In this invention the test sample comprises the ambient atmosphere of the environment together with the totality of odours or Volatile Organic Compounds emanating from the group consisting of breath samples, sweat samples, urine samples, vaginal samples, faeces samples, tissue samples, eructation samples, flatulence samples and any other samples having a bodily origin. Thus the sample taken is a holistic or full body sample taken rather than one from a single specific source in the body.

In accordance with one aspect of the present invention, there is provided apparatus comprising: (a) an air sampling unit able to take a sample of the atmosphere in the environment and to divert said sample to enter the sensor set; (b) a selected definitive sensor set comprising at least two sensors known to react to the presence of the specific odours or volatile organic compounds (VOCs) in the air sample which are known to indicate the presence of a specific disease or diseases, the air sample being taken from the environment; and (c) a processing unit comprising a pattern recognition analyser, wherein the pattern recognition analyser receives output signals of the sensor set compares them to disease —specific patterns derived from a database of response patterns of the sensor set to the breath and other bodily emissions of subjects with known diseases, wherein each of the disease —specific patterns is characteristic of a particular disease, selected from the group consisting of bacteriological, viral and parasitic diseases; and selects a closest match between the output signals of the sensor set and the disease—specific pattern and (d) a control system that triggers the sampling of the air space of the environment at pre-determined times or intervals so rendering the apparatus entirely automatic and self-contained in operation.

While laboratory instruments with high specificity and accuracy are available, they are not generally suitable for room monitoring applications because they lack physical robustness, are not automatic in operation and require highly trained operators, and typically are large in size and weight, have high power consumption requirements, and chemical reagent (gases, liquids) requirements and require fluid samples, such as breath samples, to be collected separately and manually. Such instruments are generally too expensive to be installed in multiple rooms in a building or installed in multiple locations in one large room or facility, or for use in agricultural screening and testing.

This present invention does not detect the specific bacteria, virus and/or parasite associated with a disease; rather it detects the presence of a combination of odours or Volatile Organic Compounds which are generated in the subject as a reaction to the infection. With a suitable array of sensors and appropriate processing of their reactions it is possible to build up a digital fingerprint which is specific to a single disease. With a library of such digital fingerprints stored within the apparatus it becomes possible, through a process of comparison, to identify the presence of disease even when two or more are present in one subject or when multiple subjects each have a specific but different disease. It has been found that in the case of diseases caused by viral infections the indicative combinations of odours or Volatile Organic Compounds will include, but not be limited to, chemical compounds from the aldehydes (ethanal, heptanal, octanal), ketones (acetone, butanone) and methanol (D. M. Ruszkiewicz et al., Diagnosis of COVID-19 by analysis of breath with gas chromatography-ion mobility spectrometry—a feasibility study, EClinicalMedicine (2020). In the case of diseases caused by bacterial infections the indicative combinations of odours or Volatile Organic Compounds will include, but not be limited to, chemical compounds from the alcohols (3-methyl-butanol, propan-2-ol), aldehydes (propanal, hexanal, 3-methyl-butanal) and sulphur compounds (methyl sulphide, dimethyl sulphide) group.

It further becomes possible following the outbreak of a previously unknown disease to adapt the apparatus to detect the new disease by sampling and measuring the odours or Volatile Organic Compounds emitted by subjects infected with the new disease and to determine the digital fingerprint describing the combination of odours or Volatile Organic Compounds identifying said disease. The apparatus could then have its digital library and software updated to include the new disease.

The system could be used to automatically and continuously or periodically monitor an environment including but not limited to aircraft cabins; ship and boat cabins; motor vehicle interiors; offices; homes; schools; health and medical facilities; sports and training facilities, poultry houses; cattle barns; pig (swine) pens; in each case to detect viral, bacterial and or parasitic diseases causing illness in animals and human beings. The system could be installed in rooms as a stand-alone monitor in the fashion of a smoke alarm or could be installed as part of another system such as an air-conditioning installation or an air filtering or air purifying system.

Following the detection of a disease the system could provide alerts through numerous channels including but not limited to Wi-Fi; SMS telephone messaging; Bluetooth® and other wireless communications; direct wired connections to other equipment; as well as audible and visual indicators and alarms. All the historical measurements with time and date codes could be stored in memory for subsequent review and auditing.

The identity of many disease infections is indicated by the presence of a unique group of VOCs, usually numbering five or more, in particular concentrations. These VOC emissions can be from the subject's breath; body sweat, urine, faeces and intestinal gases. Each emission may have a different group of VOCs in different concentrations but together they combine to give a whole body signature or definition of the VOC emissions of the subject. Similarly any two or more sources, say breath and body sweat, can combine to form a signature. There are previous inventions based upon the singular use of breath samples but these give poor results, unless considerable care is taken, because of the presence of high levels of water vapour and sometimes residual gases from the digestive system. These signatures can be used to specify a particular disease infection of the subject. Such signatures can be stored, usually in a digital data form, and then used for comparison with subsequent VOC samples to determine if the subject is infected with specific diseases. However there are a number of difficulties in making a practical realisation of this invention. First many of the target VOCs defining the signature can be very similar to each other. For example this signature may include several alcohol compounds and so the sensors will need to be very specific to particular organic compounds. In such cases the use of a simple hydrocarbon sensor will not be adequate as its response will be too broad. Secondly many of the target VOCs will be very low in concentrations; either by dilution in high volume environments or because their presence is swamped by the presence of other gases or vapours in the environment. An example would be a chicken house where the levels of ammonia, water vapour and carbon dioxide could be of the order of over 100 parts per million (by volume) whilst the levels of the target VOCs could be one part per billion or less. These practical limitations require a very efficient method of collecting an air sample containing the target VOCs and an array of extremely sensitive and selective sensors.

This invention collects, analyses and measures samples from an environment to determine if one or more subjects in that environment are infected with a specific disease. By way of illustration such environments can range in size from a step-in test booth to a classroom; an office; an aircraft or other transportation cabin; to a very large agricultural building.

The air sample taken would need to be representative of the air volume of the particular environment. By way of illustration; with an air sampling system drawing 100 litres of air per minute in an environment of total volume 100 cubic metres; would take 16.7 hours to completely sample the air in that environment. It follows therefore that a rapid measurement of the VOCs in that environment would require a higher rate of airflow or the use of multiple air samplers or complete measurement systems spread across the environment.

It should be noted that high rates of airflow are not compatible with most gas sensors as the VOCs have insufficient time to settle on the sensor's sensitive surfaces before they are blown away. One solution is an intermediate stage of VOC collection by using an adsorbent material in the air flow; this material being chosen or engineered to be particularly sticky to the target VOCs. After the air sample is taken the adsorbent material can be heated to release the target VOCs which are then directed over the sensor array. The temperature range of the heater is chosen to specifically release the target VOCs by evaporation rather than every VOC.

This invention is to collect the totality of VOCs in an environment and to selectively identify and measure those target VOCs which form the signature of a specific disease infection. Although the invention will work with one single subject it is particularly effective in identifying one or more infected subjects in an environment occupied by multiple subjects. It is desirable for a practical implementation of the invention to include a VOC adsorber in the air sampling system and which incorporates a heater whose temperature can be set to release by evaporation the target VOCs amongst the multitude of VOCs that may have been adsorbed.

It is desirable for a practical implementation of the invention to include an array of VOC sensors of very high sensitivity (typically 5 parts per billion by volume) and specificity.

In accordance with a first aspect of the present invention, there is provided apparatus for detecting the presence in an enclosed environment of a subject or subjects infected with viral and/or bacterial disease or diseases, the apparatus comprising:
(a) an air sampling unit able to take an air sample of the atmosphere in the enclosed environment and to divert said sample for sensing;
(b) a selected definitive sensor set comprising at least two sensors reactive to the presence of specific odours or Volatile Organic Compounds (VOCs) in the air sample taken from the environment;
(c) a processing unit comprising a pattern recognition analyser, wherein the pattern recognition analyser receives output signals of the sensor set, compares them to disease-specific patterns derived from a database of response patterns of the sensor set exposed to the totality of the bodily emissions of subjects with known disease or diseases, wherein each of the disease-specific patterns is characteristic of a particular disease, selected from bacteriological, viral and parasitic diseases, and selects a closest match between the output signals of the sensor set and the disease-specific pattern; and
(d) a control system that triggers the sampling of the air space of the environment at pre-determined times or intervals for rendering the apparatus entirely automatic and self-contained in operation,

wherein the air sampling unit comprises: a surface for capturing VOCs from the air sample; and a heater for heating the surface to release captured VOCs when diverting the air sample for sensing.

The surface may be one of a plurality of surfaces, each for capturing VOCs from the air sample.

The at least two sensors of the senor set may be configured to detect different Volatile Organic Compounds (VOCs) associated with the diseases of interest. For example, a first sensor may be configured to detect a first VOC, and a second sensor may be configured to detect a second VOC which is different to the first VOC. The sensor set may comprise at least five sensors, each configured to detect different Volatile Organic Compounds (VOCs).

For example, the sensor set may have twelve such sensors, or even twenty-four such sensors. The apparatus relies upon the fact that with an array of sensors each one is different and intended to be specific to one of the target VOCs in the digital fingerprint of the particular disease. If the “fingerprint” of the specific disease includes six target VOCs, say two alcohols, a ketone, two aldehydes and an ester, the sensor array should have at least six different sensors each one designed to be specific to one of the target VOCs so that all six (in this case) target VOCs can be detected.

In one arrangement, for example, an array of twenty-four sensors may be provided to enable perhaps at least four or maybe more diseases to be detected depending upon the number of specific VOCs in their individual fingerprint. Sometimes there will be an overlap where disease A has a fingerprint of VOCs M, N, O, P & Q whereas Disease B has a fingerprint of VOCs W, X, Y, Z, & Q., meaning that because of the partial commonality some sensors might be shared, sensing VOCs common the both Disease A and Disease B.

In the apparatus, at least one sensor may be selected from the group consisting of surface acoustic wave sensors, quartz crystal resonators, metal oxide sensors, dye-coated fibre optic sensors, micro-machined cantilever arrays, composites having regions of conducting material and regions of insulating organic material, composites having regions of conducting material and regions of conducting or semi-conducting organic material, chemically-sensitive resistor or capacitor film, semi-conducting polymer sensors, metal-oxide-semiconductor field effect transistors, and bulk organic conducting polymeric sensors.

In the apparatus, the air sample tested may be configured to comprise the ambient atmosphere of the enclosed environment together with the totality or combined total of the odours or Volatile Organic Compounds (VOCs) selected from the group consisting of breath samples, sweat samples, urine samples, vaginal samples, faeces samples, tissue samples; eructation samples, flatulence samples and any other samples having a bodily origin.

In the apparatus, the processing unit may be configured to analyse data by comparing the data to a database containing data profiles from a plurality of detectable signals and identifying the at least one specific odour or Volatile Organic Compound present in the sample thereby characterizing the disease or combinations of diseases.

In the apparatus, the air sampling unit may be configured to pass air of the enclosed environment over the surface. For example, the air sampling unit may further comprise a fan for directing air from the atmosphere in the enclosed environment towards the surface. The surface may be housed in an open-ended conduit, with the fan positioned to direct air from the atmosphere in the enclosed environment through the conduit. In the apparatus, the air sampling unit may be configured to pass a volume of air of the enclosed environment over the surface which is greater than the volume of the air sample diverted for sensing. For example, the air sampling unit may be configured to pass a volume of air of the enclosed environment over the surface which is at least 100 times greater than the volume of the air sample diverted for sensing.

In the apparatus, the surface for capturing VOCs from the air sample may be configured to adsorb or absorb VOCs from the air sample. For example, the surface for capturing VOCs from the air sample may be part of a material selected from the group consisting of a nano-porous material, a microporous material, a chemically reactive material, a nonporous material and combinations thereof.

In the apparatus, at least one of the at least two sensors in the sensor set has a sensitivity of at least 5 parts per billion by volume, e.g. 3 parts per billion by volume, or even 1 part per billion by volume.

In the apparatus, the air sampling unit may be configured to heat the heater to a temperature to release a specific VOC captured by the surface. More than one surface may be provided, and each may have a respective heater. The heaters may be configured to heat their respective surfaces to different temperatures. In this way, a first heater may be configured to release a first specific VOC captured by the surface associated with the first heater, and a second heater may be configured to release a second specific VOC (different to the first specific VOC) captured by the surface associated with the second heater.

The apparatus may further comprise an air filter unit configured to remove Volatile Organic Compounds from ambient air and provide purified air for cleaning and purging the sensor set before sensing the air sample.

The apparatus may be incorporated into an air purifying system of a type used to filter out bacterial and/or viral infection, with the processing unit being used to determine whether the air purifying system is working effectively, e.g. by comparing the output signals of the sensor set before and after filtration.

In accordance with another aspect of the present invention, there is provided a method of diagnosing, screening or monitoring the presence of a viral, bacterial or parasitic disease or diseases in a test subject in a non-invasive manner by sampling the atmosphere around the subject in an enclosed environment, the method comprising the steps of:
(a) providing apparatus according to the first aspect of the present invention (or embodiments thereof);
(b) capturing on the surface of the air sampling unit VOCs from an air sample, the VOCs being selected from the group consisting of breath samples, urine samples, vaginal samples, faeces samples, tissue samples, eructation samples, flatulence samples and any other samples having a bodily origin of the test subject;
(c) heating the surface to release captured VOCs and diverting the released VOCs to the sensor set;
(d) measuring the output signals of the sensor set upon exposure to the said sample;
(e) comparing the output signals using a pattern recognition analyser to the database-derived disease-specific patterns; and
(e) selecting the closest match between the output signals of the sensor set and the database-derived disease-specific patterns.

The method may further comprise applying a tolerance between the output signals of the sensor set and the database-derived disease-specific patterns, and indicating a Pass or a Fail (Infected or Non-Infected) condition in dependence upon whether the tolerance is exceeded or not.

The method may further comprise the step of passing air from the atmosphere around the subject over the surface to concentrate the Volatile Organic Compounds captured on the surface. For example, passing air from the atmosphere around the subject over the surface may concentrate the Volatile Organic Compounds captured on the surface by a factor of at least 10.

In the method, the enclosed environment may be selected from the group consisting of: an aircraft cabin; a ship or boat cabin; a motor vehicle interior; an office; a home; a school; a health or medical facility; a sports or training facility a poultry house; a cattle barn; a pig (swine) pen, and a test booth (e.g. for testing one or more persons in an airport terminal).

The method may further comprise adding a disease-specific pattern characterising a disease or diseases not previously included in the database of response patterns of the sensor set to the bodily emissions of subjects with known diseases.

In the method, the Volatile Organic Compounds of interest when monitoring for a disease caused by a viral infection may include chemical compounds selected from, but not limited to, the aldehydes, ketones and alcohols groups, and the Volatile Organic Compounds of interest when monitoring for a disease caused by a bacterial infection may include chemical compounds selected from, but not limited to, the alcohols, aldehydes and sulphides groups.

It is well known that certain volatile compounds can be detected on the breath and that such volatiles can be indicative of certain diseases or conditions. This invention does not attempt to measure the bacteria or viruses which cause the disease infection. Instead it characterises the disease by means of the changes in the biochemistry of the subject's body; these changes being revealed in the pattern of odours or VOCs being emitted in the breath, sweat or other bodily odours of the subject. By way of example, it is recognised that the smell of acetone on the breath is indicative of ketosis, and that this can be related to diabetes mellitus. It is also well known that alcohol on the breath is indicative of blood alcohol levels. The technique of detecting diseases by measuring and analysing and making comparison with the digital fingerprints of mixtures of odours or Volatile Organic Compounds is well established in the prior art with several inventions using breath samples from subjects.

Examples of this prior art include Neel (1994) U.S. Pat. No. 5,356,594, Satterfield (2014) U.S. Pat. Application US 2014/0276100 A1, Philips (2001) U.S. Pat. No. 6,312,390, Philips (2001) U.S. Pat. No. 6,221,026, Sood (2014) U.S. Pat. Application US 2014/0127326 A1 and Wang (2004) U.S Pat. Application US 2004/0137637 A1.

The present invention collects a sample of the odours or VOCs onto an adsorber. This is a material which has been selected for its ability to adsorb and temporarily retain the target odours or VOCs emitted by the subject. This adsorber could take the form of a body swab; a piece of clothing worn for a period of time; or a breath sample. After use the adsorbed sample is placed in a container attached to a VOC sampler and analyser. The sample is heated to a temperature between 30 and 100 degrees Celsius which action releases the odours or Volatile Organic Compounds which have been retained in the sample adsorber from the subject's breath or body odours.

The base system comprises: (a) a container for holding the sample adsorber, said container having an air inlet and an air outlet to the sampling unit; the assembly being heated by a temperature controlled heater; (b) an air sampling unit able to take a sample of the air from the container and to divert said sample to enter the sensor set; (c) a selected definitive sensor set comprising at least two sensors known to react to the presence of the specific odours or Volatile Organic Compounds (VOCs) in the air sample which are known to indicate the presence of a specific disease or diseases, and (d) a processing unit comprising a pattern recognition analyser, wherein the pattern recognition analyser receives output signals of the sensor set compares them to disease-specific patterns derived from a database of response patterns of the sensor set to the breath or other body sweat emissions of subjects with known diseases, wherein each of the disease-specific patterns is characteristic of a particular disease, both bacteriological or viral; and selects a closest match between the output signals of the sensor set and the disease-specific pattern.

The sensitivity of the system can be enhanced by using for the sampling a piece of material known to efficiently absorb odours or Volatile Organic Compounds. Suitable commercially available absorbent materials include but are not limited to, Tenax TA, Tenax GR, Carbotrap, Carbopack B and C. Carbotrap C, Carboxen, Car bosieve SIII, Proapak, Spherocarb, and combinations thereof; preferred adsorbent combinations include, but are not limited to, Tenax GR and Carbopack B: Carbopack B and Carbosieve SIII; and Carbopack C and Carbopack Band Carbosieve SIII or Carboxen 1000. Those skilled in the art will know of other suitable absorbent materials.

It further becomes possible following the outbreak of a previously unknown disease to adapt the apparatus to detect the new disease by sampling and measuring the odours or Volatile Organic Compounds emitted by subjects infected with the new disease and to determine the digital fingerprint describing the combination of odours or Volatile Organic Compounds identifying said disease. The apparatus could then have its digital library and software updated to include the new disease. All the historical measurements with time and date codes could be stored in memory for subsequent review and auditing.

In accordance with a second aspect of the present invention, there is provided apparatus for detecting a viral, bacterial or parasitic disease in a subject, comprising:
(a) a housing for receiving a material contaminated by exposure to the subject's breath or sweat, the housing comprising a heater configured to heat the material when received therein to a controlled temperature between 30 and 100 degrees Celsius;
(b) an air sampling unit able to take a sample of the odours or Volatile Organic Compounds emitted by the material heated inside said housing and to divert said sample for sensing;
(c) a selected definitive sensor set comprising at least two sensors reactive to the presence of specific odours or Volatile Organic Compounds (VOCs) in the sample taken from the material; and
(c) a processing unit comprising a pattern recognition analyser, wherein the pattern recognition analyser receives output signals of the sensor set, compares them to disease-specific patterns derived from a database of response patterns of the sensor set exposed to the breath or sweat samples of subjects with known disease, wherein each of the disease-specific patterns is characteristic of a particular disease, selected from bacteriological, viral and parasitic diseases; and selects a closest match between the output signals of the sensor set and the disease-specific pattern.

The at least two sensors of the senor set may be configured to detect different Volatile Organic Compounds (VOCs) associated with the diseases of interest. For example, a first sensor may be configured to detect a first VOC, and a second sensor may be configured to detect a second VOC which is different to the first VOC. The sensor set may comprise at least five sensors, each configured to detect different Volatile Organic Compounds (VOCs). For example, the sensor set may have twelve such sensors, or even twenty-four such sensors. The apparatus relies upon the fact that with an array of sensors each one is different and intended to be specific to one of the target VOCs in the digital fingerprint of the particular disease. If the “fingerprint” of the specific disease includes six target VOCs, say two alcohols, a ketone, two aldehydes and an ester, the sensor array should have at least six different sensors each one designed to be specific to one of the target VOCs so that all six (in this case) target VOCs can be detected.

In one arrangement, for example, an array of twenty-four sensors may be provided to enable perhaps at least four or maybe more diseases to be detected depending upon the number of specific VOCs in their individual fingerprint. Sometimes there will be an overlap where disease A has a fingerprint of VOCs M, N, O, P & Q whereas Disease B has a fingerprint of VOCs W, X, Y, Z, & Q., meaning that because of the partial commonality some sensors might be shared, sensing VOCs common the both Disease A and Disease B.

In the apparatus, at least one sensor may be selected from the group consisting of surface acoustic wave sensors, quartz crystal resonators, metal oxide sensors, dye-coated fibre optic sensors, micro-machined cantilever arrays, composites having regions of conducting material and regions of insulating organic material, composites having regions of conducting material and regions of conducting or semi-conducting organic material, chemically-sensitive resistor or capacitor film, semi-conducting polymer sensors, metal-oxide-semiconductor field effect transistors, and bulk organic conducting polymeric sensors.

In the apparatus, the housing for receiving the material contaminated by exposure to the subject's breath or sweat may be removable and/or disposable to enhance the biosecurity of the system. In the apparatus, the heater may be configured to heat the material to a temperature to release a specific VOC.

In the apparatus, the data may be analysed by comparing the data to a database containing data profiles from a plurality of detectable signals and identifying the at least one specific odour or Volatile Organic Compound present in the sample thereby characterizing the disease or combinations of diseases.

In the apparatus, the disease or diseases in the subject may be of bacteriological or viral origin, and the processing unit may be configured to differentiate between a disease of bacteriological origin and one of viral infection by comparing the results with a library of stored VOC digital fingerprints.

In the apparatus, the database may comprise response patterns of the sensor set when sampling odours or Volatile Organic Compounds emanating from the group consisting of breath samples and body sweat samples having a bodily origin of subjects suffering from a known disease.

The apparatus may further comprise an air filter unit configured to remove Volatile Organic Compounds from ambient air and provide purified air for cleaning and purging the sensor set before sensing the air sample.

In the apparatus, at least one of the at least two sensors in the sensor set may have a sensitivity of at least 5 parts per billion by volume.

The apparatus may further comprise an air filter unit, such as a capsule of adsorbent material carbon granules or carbon impregnated cloth, said air filter unit being inserted ahead of the air outlet to limit the risk of releasing any infected material into the general atmosphere.

In accordance with another aspect of the present invention, there is provided a method of diagnosing, screening or monitoring the presence of a viral, bacterial and/or parasitic disease or diseases in a test subject, the method comprising the steps of:
(a) providing apparatus according to the second aspect of the present invention (or embodiments thereof);
(b) inserting into the housing a material contaminated by exposure to the subject's breath or sweat;
(c) heating the material in the housing to a controlled temperature between 30 and 100 degrees Celsius to release captured VOCs and diverting the released VOCs to the sensor set;
(d) measuring the output signals of the sensor set upon exposure to the said sample;
(e) comparing the output signals using a pattern recognition analyser to the database-derived disease-specific patterns; and
(e) selecting the closest match between the output signals of the sensor set and the database-derived disease-specific patterns.

The method may further comprise applying a tolerance between the output signals of the sensor set and the database-derived disease-specific patterns, and indicating a Pass or a Fail (Infected or Non-Infected) condition in dependence upon whether the tolerance is exceeded or not.

In the method, the material may be configured to adsorb or absorb VOCs from the subject's breath or sweat. The material may be at least part of a face mask worn by the subject or clothing worn by the subject.

The method may further comprise collecting a breath sample using a disposable device comprising a tube coupled to a frame carrying a panel of the material configured to adsorb or absorb air blown through the tube by the subject.

The method may further comprise adding a disease-specific pattern characterising a disease or diseases not previously included in the database of response patterns of the sensor set to the bodily emissions of subjects with known diseases.

In the method, the Volatile Organic Compounds of interest when monitoring for a disease caused by a viral infection include chemical compounds may be selected from, but not limited to, the aldehydes, ketones and alcohols groups, and the Volatile Organic Compounds of interest when monitoring for a disease caused by a bacterial infection may include chemical compounds selected from, but not limited to, the alcohols, aldehydes and sulphides groups.

The method may further comprise storing the material in a sealed container before step (b), possibly by refrigerating the sealed container to maintain a temperature of below 10 degrees Celsius.

### A BRIEF DESCRIPTION OF THE DRAWINGS

FIG. 1 is a block diagram of the components of a system in accordance with the invention that includes several optional components;

FIG. 2 is a block diagram of components of a simplified system in accordance with the invention;

FIGS. 3A and 3B are diagrammatic perspective views of two free standing monitoring station systems each in accordance with the invention;

FIG. 4 is a diagrammatic view of a wall or ceiling mounted monitoring station in accordance with the invention;

FIG. 5 is a block diagram showing the system incorporated into an air-conditioning or air-purifying or air-filtration system in accordance with the invention;

FIG. 6 shows the typical responses of a sensor set or array when exposed to the full body odours or Volatile Organic Compounds firstly from subjects not infected with the disease and secondly from subjects infected with the disease;

FIG. 7 is a diagrammatic view of a screening booth incorporating the apparatus in accordance with the invention;

FIG. 8 is a block diagram of the components of a system in accordance with the invention that includes several optional components;

FIG. 9 is a diagrammatic perspective view of a portable handheld monitoring unit system in accordance with the invention; and

FIG. 10 is a diagrammatic perspective view of a Breath Sampling device.

### DETAILED DESCRIPTION OF THE INVENTION

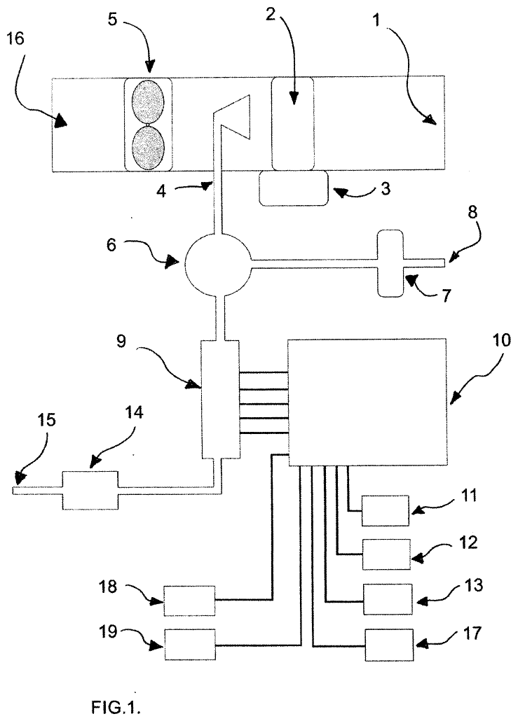

FIG. 1 illustrates a preferred embodiment of the apparatus. This includes an air inlet 1 open to the atmosphere of the environment; an absorber 2 and associated heater 3 for the pre-concentrator system; a suction fan 5 which pulls the air sample into the apparatus and an exhaust outlet 16 which returns the air to the atmosphere. A pipe 4 routes the air sample to a diverter valve 6. This valve has a changeover function and can connect the sensor set 9 to the sample pipe 4 or to the fresh air pipe 8; via the optional air filter 7. This air is used to purge or clean the sensor set after each sampling and measuring sequence. The air is pulled through the sensor set by the air pump 14 which exhausts into outlet 15. The sensor set 9 is connected to the electronic processor system 10 which is also connected to the optional display 11; the optional control panel 12; the electrical power supply 13; the optional wireless connection 17; a wired connection 18 to an external computer or other apparatus and an optional audible or visual alarm 19 to provide an alert following detection of a disease.

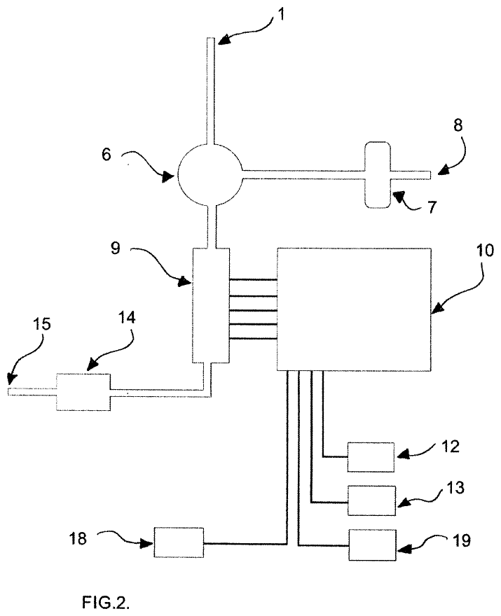

FIG. 2 illustrates an embodiment of the apparatus. This includes an air inlet 1 open to the atmosphere of the environment and which routes the air sample to a diverter valve 6. This valve has a changeover function and can connect the sensor set 9 to the air sample from air inlet 1 or to the fresh air pipe 8; via the optional air filter 7. This air is used to purge or clean the sensor set 9 after each sampling and measuring sequence. The air is pulled through the sensor set by the air pump 14 which exhausts into outlet 15. The sensor set 9 is connected to the electronic processor system 10 which is also connected to the optional control panel 12; the electrical power supply 13; and a wired connection 18 to an external computer or other apparatus and an optional audible or visual alarm 19 to provide an alert following detection of a disease.

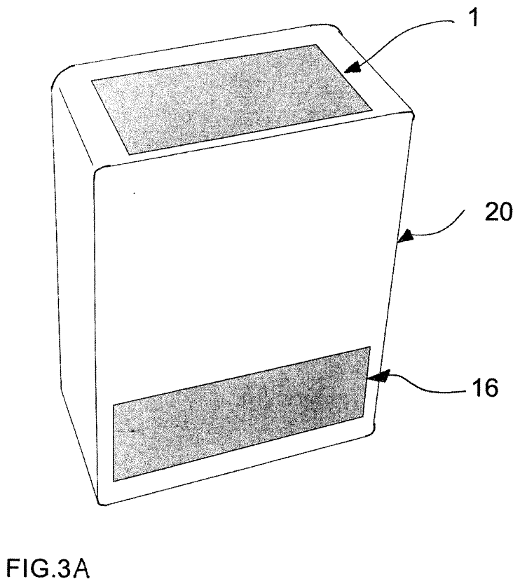

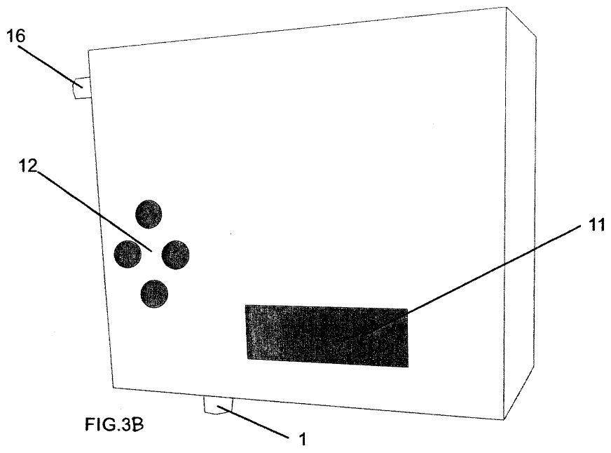

FIG. 3A. illustrates a wall mounted embodiment of the invention. Within the housing 20 there could typically be the components shown in FIG. 2. Visible components shown are the air intake 1 and the outlet 16. FIG. 3B illustrates an alternative roof or wall mounted embodiment of the invention in the manner of a traditional smoke alarm. Within the housing 20 there could typically be the components shown in FIG. 2. Visible components shown are the air intake 1, control panel 12, display 11 and the outlet 16.

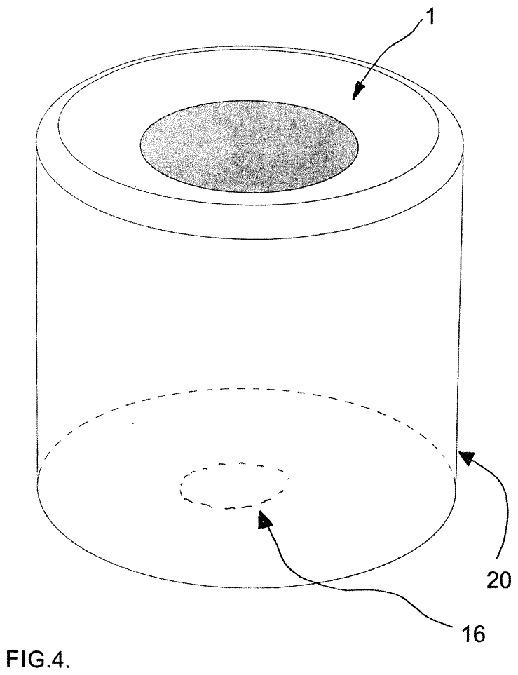

FIG. 4. illustrates a ceiling or wall mounted embodiment of the invention in the manner of a traditional smoke alarm. Within the housing 20 there could typically be the components shown in FIG. 2. Visible components shown are the air intake 1 and the outlet 16.

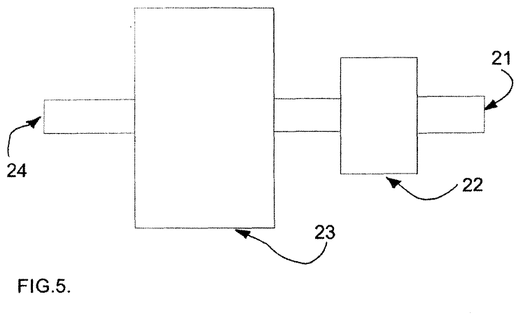

FIG. 5. is a block diagram illustrating how the invention might be incorporated into an air-conditioning system. This embodiment could give a simple method of extracting an air sample for measurement and analysis from the room or environment being monitored. The air-conditioning intake 21 is interrupted by an air monitoring system 22 typically comprising the apparatus shown in FIG. 1. after which the air flow continues into the air-conditioning equipment 23 and to the outlet 24.

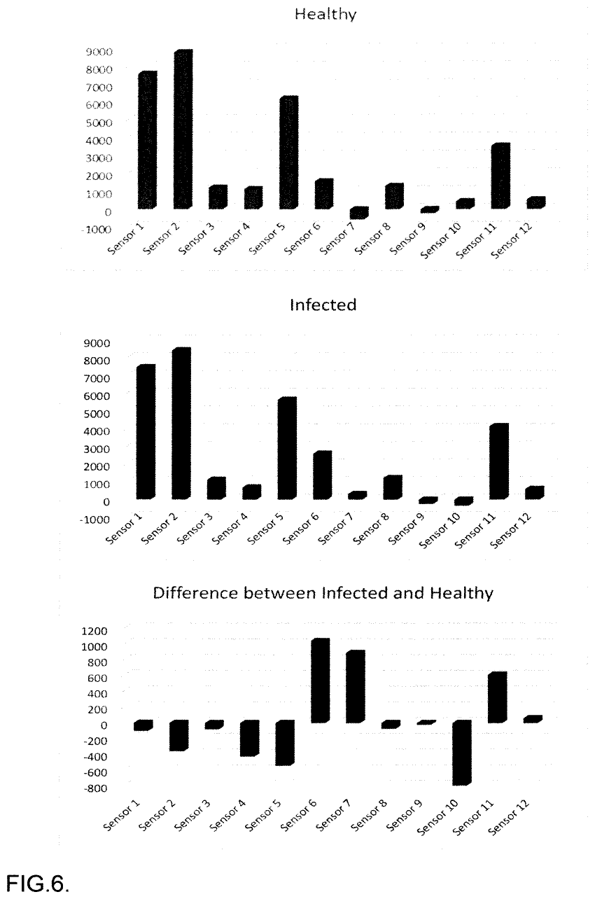

FIG. 6. Shows the amplitude responses of a sensor set or array when (a) exposed to the atmosphere containing the odours or Volatile Organic Compounds emitted from subjects not infected with the disease (Healthy), (b) when exposed to the atmosphere containing the odours or Volatile Organic Compounds emitted from subjects infected with the disease (Infected) and c) the difference between the Infected and Healthy. These illustrative results were taken with the specific disease Campylobacter Infection. In this case the sensor responses are differentiated by the maximum amplitude deviation from baseline, with the difference between the Infected and healthy the criteria for analysis, but there are many other more complex pattern recognition techniques which might be implemented depending on the application. Such techniques include, but are not limited to, K-nearest neighbour (KNN), Canonical Discriminate Analysis (CDA), Soft Independent Modelling of Class Analogy (SIMCA), probabilistic neural network (PNN), artificial neural network (ANN), support vector machine (SVM), Linear Discriminant Analysis (LDA) and Fisher Linear Discriminate (FLD).

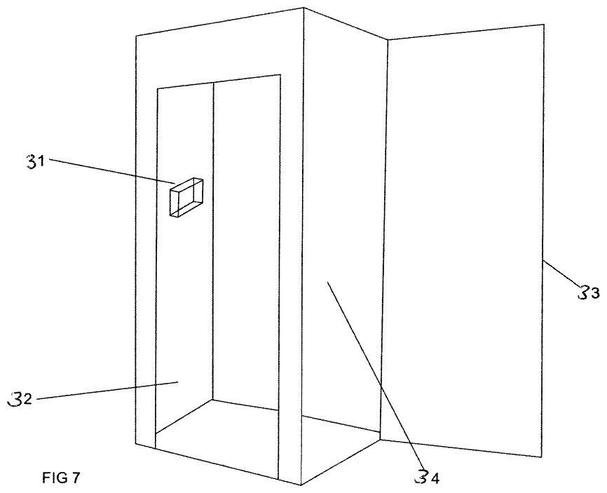

FIG. 7. is a block diagram illustrating how the invention might be incorporated into a screening booth of a type that might be used at an airport or the entrance to an office block or in other applications. This embodiment could give a simple method of extracting an air sample for measurement and analysis from the interior of the booth. The booth could consist of an interior space 34 where the subject would stand; a window 32; a door 33 and the apparatus shown in FIG. 1 mounted in housing 31.

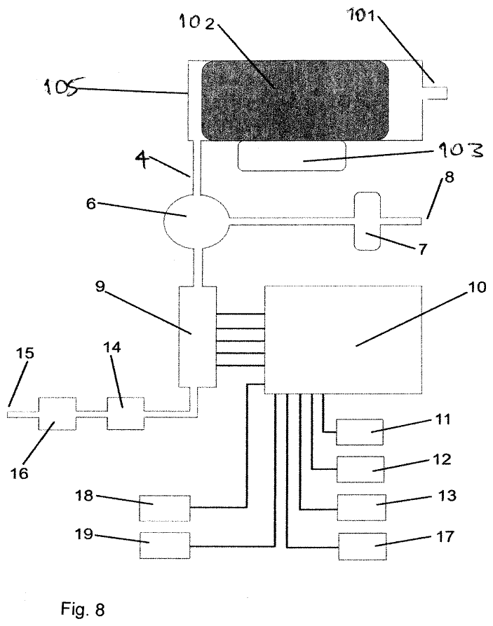

FIG. 8 illustrates another embodiment of the apparatus (features in common with the embodiment of FIG. 1 share the same reference numbers). This includes an air inlet 101 open to the atmosphere of the environment; an absorbed sample 102 and associated heater 103; and a container 105 for housing the sample. A pipe 4 routes the air sample from the container 105 to a diverter valve 6. This valve has a changeover function and can connect the sensor set 9 to the sample pipe 4 or to the fresh air pipe 8; via the optional air filter 7. This air is used to purge or clean the sensor set after each sampling and measuring sequence. The air is pulled through the sensor set by the air pump 14 which exhausts into outlet 15. This outlet may incorporate an optional air filter 16 containing suitable absorbent material to limit the risk of releasing any infected material into the general atmosphere. The sensor set 9 is connected to the electronic processor system 10 which is also connected to the optional display 11; the optional control panel 12; the electrical power supply 13; the optional wireless connection 17; a wired connection 18 to an external computer or other apparatus and an optional audible or visual alarm 19 to provide an alert following detection of a disease.

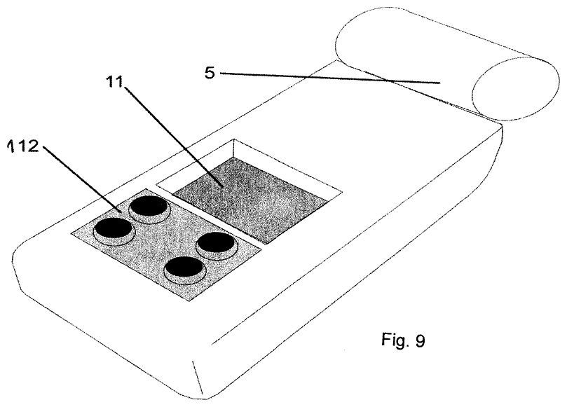

FIG. 9 is a diagrammatic perspective view of a portable handheld monitoring unit system in accordance with the invention; within the housing 112 there could typically be the components shown in FIG. 1.

Referring back to FIG. 6 which shows the amplitude responses of a sensor set or array when (a) exposed to the atmosphere containing the odours or Volatile Organic Compounds emitted from subjects not infected with the disease (Healthy), (b) when exposed to the atmosphere containing the odours or Volatile Organic Compounds emitted from subjects infected with the disease (Infected) and c) the difference between the Infected and Healthy. These illustrative results were taken with the specific disease Campylobacter Infection. In this case the sensor responses are differentiated by the maximum amplitude deviation from baseline, with the difference between the Infected and healthy the criteria for analysis, but there are many other more complex pattern recognition techniques which might be implemented depending on the application. Such techniques include, but are not limited to, K-nearest neighbour (KNN), Canonical Discriminate Analysis (CDA), Soft Independent Modelling of Class Analogy (SIMCA), probabilistic neural network (PNN), artificial neural network (ANN), support vector machine (SVM), Linear Discriminant Analysis (LDA) and Fisher Linear Discriminate (FLD).

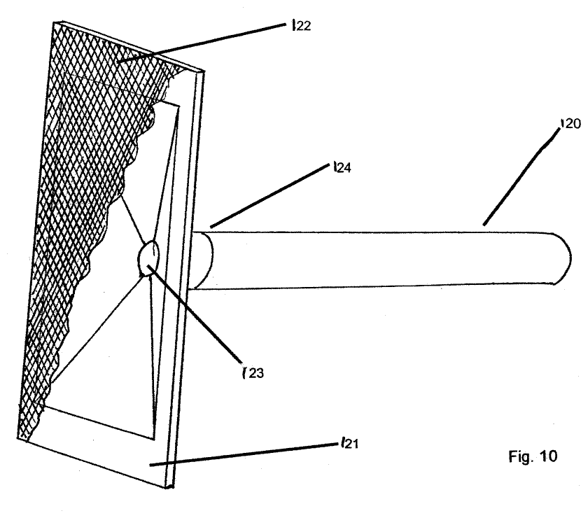

FIG. 10 is a diagrammatic perspective view of a Breath Sampling device. The device comprises a tube 120 through which the breath is blown through an entry hole 123 into a housing 121 which is sealed by a piece of adsorbent material 122 chosen for its ability to adsorb and temporarily retain the target odours or VOCs. After use the tube is removed at joint 124 for disposal and the housing is retained for measurement and analysis. The tube can be made of plastic or other material such as paper, which in the style of drinking straws give a recyclable option. The adsorbent material includes commercially available absorbent materials but not limited to, Tenax TA, Tenax GR, Carbotrap, Carbopack B and C. Carbotrap C, Carboxen, Carbosieve SIII, Proapak, Spherocarb, and combinations thereof; preferred adsorbent combinations include, but are not limited to, Tenax GR and Carbopack B: Carbopack B and Carbosieve SIII; and Carbopack C and Carbopack Band Carbosieve SIII or Carboxen 1000. Those skilled in the art will know of other suitable absorbent materials.

Although the invention has been described in detail with reference to the presently preferred embodiments, those of ordinary skill in the art will appreciate that various modifications can be made without departing from the invention.

There is therefore provided in accordance with aspects of the present invention:

Inventive concept 1. An automatically operating apparatus which will detect the presence in an environment of a subject or subjects infected with viral & bacterial disease or diseases, causing illness in animals and human beings, by measuring and analysing and making comparison with the digital fingerprints of mixtures of the totality, or a combination of any two, of the odours or Volatile Organic Compounds in the environment atmosphere and emanating from the group consisting of breath samples, sweat samples, urine samples, vaginal samples, faeces samples, tissue samples; eructation samples, flatulence samples and any other samples having a bodily origin, and, the apparatus comprising:
(a) an air sampling unit able to take a sample of the atmosphere in the environment and to divert said sample to enter the sensor set;
(b) a selected definitive sensor set comprising at least two sensors reactive to the presence of specific odours or Volatile Organic Compounds (VOCs) in the air sample taken from the environment;
(c) a processing unit comprising a pattern recognition analyser, wherein the pattern recognition analyser receives output signals of the sensor set; compares them to disease —specific patterns derived from a database of response patterns of the sensor set exposed to the totality of the bodily emissions of subjects with known disease or diseases, wherein each of the disease—specific patterns is characteristic of a particular disease, both bacteriological or viral; and selects a closest match between the output signals of the sensor set and the disease—specific pattern;
(d) a control system that triggers the sampling of the air space of the environment at pre-determined times or intervals so rendering the apparatus entirely automatic and self-contained in operation; and
(e) a housing.

Inventive concept 2. The method of inventive concept 1, wherein at least one other sensor is selected from the group consisting of surface acoustic wave sensors, quartz crystal resonators, metal oxide sensors, dye-coated fibre optic sensors, micro-machined cantilever arrays, composites having regions of conducting material and regions of insulating organic material, composites having regions of conducting material and regions of conducting or semi-conducting organic material, chemically-sensitive resistor or capacitor film, semi-conducting polymer sensors, metal-oxide-semiconductor field effect transistors, and bulk organic conducting polymeric sensors. Such sensors having a sensitivity of 5 parts per billion by volume or higher.

Inventive concept 3. The method of inventive concept 1, wherein said test sample comprises the ambient atmosphere of the environment together with the totality or combined total of the odours or Volatile Organic Compounds emanating from the group consisting of breath samples, sweat samples, urine samples, vaginal samples, faeces samples, tissue samples; eructation samples, flatulence samples and any other samples having a bodily origin or the combination of any two from the group.

Inventive concept 4. The method of inventive concept 1, wherein the data is analysed by comparing the data to a database containing data profiles from a plurality of detectable signals and identifying the at least five specific odours or Volatile Organic Compounds present in the sample thereby characterizing the disease or combinations of diseases.

Inventive concept 5. The method of inventive concept 1, wherein the disease or diseases causing the illness in the subject (animal or human being) is of bacteriological or viral origin. Inventive concept 6. The system according to inventive concept 1 wherein the database comprises response patterns of the sensor set when sampling odours or Volatile Organic Compounds emanating from the group consisting of breath samples, urine samples, vaginal samples, faeces samples, tissue samples; eructation samples, flatulence samples and any other samples having a bodily origin of multiple subjects suffering from a known disease.

Inventive concept 7. The system according to inventive concept 1, with a method of diagnosing, screening or monitoring the presence of a disease or diseases in a test subject in a non-invasive manner by sampling the atmosphere around the subject, the method comprising the steps of: (a) providing a system according to claim 1; (b) exposing the sensor set to the combined odours or Volatile Organic Compounds emanating from any two or more of the group consisting of breath samples, urine samples, vaginal samples, faeces samples, tissue samples; eructation samples, flatulence samples and any other samples having a bodily origin of the test subject; (c) measuring the output signals of the sensor set upon exposure to the said sample; (d) comparing the output signals using a pattern recognition analyser to the database—derived disease—specific patterns; and (e) selecting the closest match between the output signals of the sensor set and the database—derived disease specific patterns and applying a tolerance between the output signals of the sensor set and the database—derived disease specific patterns, to indicate a Pass or a Fail (Infected or Non-Infected) condition.

Inventive concept 8. The method according to inventive concept 1 further comprising the step of concentrating the odours or Volatile Organic Compounds contained in the air sample by passing the sample over or through an absorbent material for a longer period. The absorbent material of the fluid concentrator can be, but is not limited to, a nano-porous material, a microporous material, a chemically reactive material, a nonporous material and combinations thereof and in some instances, the absorbent material can concentrate the odours or Volatile organic compounds by a factor of between 10 to about 100. The material will be chosen to particularly adsorb those VOCs which are chosen or targeted for detection. Once the sample is concentrated, it can be desorbed by heating the absorbent material. The temperature range of the heating process being chosen to release principally the target or chosen VOCs in the multiplicity that will have been adsorbed. Suitable commercially available absorbent materials include but are not limited to, Tenax TA, Tenax GR, Carbotrap, Carbopack B and C. Carbotrap C, Carboxen, Car bosieve SIII, Proapak, Spherocarb, and combinations thereof; preferred adsorbent combinations include, but are not limited to, Tenax GR and Carbopack B: Carbopack B and Carbosieve SIII; and Carbopack C and Carbopack Band Carbosieve SIII or Carboxen 1000. Those skilled in the art will know of other suitable absorbent materials.

Inventive concept 9. The system according to inventive concept 1 with the addition of a wireless communication device, including but not limited to Wi-Fi; Bluetooth® and Telephone SMS messaging, to enable a warning signal to be transmitted in the event of a disease or diseases being detected in the environment which is being monitored.

Inventive concept 10. The system according to inventive concept 1 with the addition of an air filter unit, including but not limited to a capsule of carbon granules or carbon impregnated cloth, and being capable of adsorbing odours and Volatile Compounds, said air filter unit purifying the air used to clean and purge the sensor set before the next sampling and measurement sequence.

Inventive concept 11. The system according to inventive concept 1 being used to automatically and continuously or periodically monitor an environment including but not limited to testing booths, aircraft cabins; ship and boat cabins; motor vehicle interiors; offices; homes; schools; health and medical facilities; sports and training facilities, poultry houses; cattle barns; pig (swine) pens; in each case to detect a subject or subjects infected with a viral & bacterial disease or diseases causing illness in animals and human beings.

Inventive concept 12. The system according to inventive concept 1 being electrically powered by current derived from a mains electricity supply; by current from a primary or a rechargeable battery; by current from a large value capacitor.

Inventive concept 13. The system according to inventive concept 1 with the provision of a computer or other memory and being capable of recording every measurement taken by every sensor in the sensor set together with date and time records; these records being downloaded through an electrical or wireless connection to said apparatus.

Inventive concept 14. The method of inventive concept 1, wherein the disease-specific patterns in the database can be added to with new disease-specific patterns characterising a disease or diseases not previously included in the database of response patterns of the sensor set to the bodily emissions of subjects with known diseases.

Inventive concept 15. The method of inventive concept 1, wherein the cases of diseases caused by viral infections the indicative combinations of odours or Volatile Organic Compounds will include, but not be limited to, chemical compounds from the aldehydes, ketones and alcohols group and in the case of diseases caused by bacterial infections the indicative combinations of odours or Volatile Organic Compounds will include, but not be limited to, chemical compounds from the alcohols, aldehydes and sulphides group.

Inventive concept 16. The system according to inventive concept 1 being capable of operation by remote control by means of electrical connection or wireless communication and allowing operational control; changes to settings; transfer of data and updating and additions to the database of response patterns of the sensor set to the bodily emissions of subjects with known diseases.

Inventive concept 17. An apparatus which will detect a viral or bacterial disease in a subject by measuring and analysing and making comparison with the digital fingerprints of the odours or Volatile Organic Compounds released from the subject's breath absorbed into a material; and, the system comprising: (a) a housing into which the sample can be inserted also containing a heater whose temperature can be controlled between 30 and 100 degrees Celsius, (b) an air sampling unit able to take a sample of the odours or Volatile Organic Compounds emitted by the sample heated inside said container and to divert said sample to enter the sensor set; (b) a selected definitive sensor set comprising at least two sensors reactive to the presence of specific odours or Volatile Organic Compounds (VOCs) in the air sample taken from the adsorber; and (c) a processing unit comprising a pattern recognition analyser, wherein the pattern recognition analyser receives output signals of the sensor set; compares them to disease-specific patterns derived from a database of response patterns of the sensor set exposed to the breath samples of subjects with known disease, wherein each of the disease-specific patterns is characteristic of a particular disease, both bacteriological or viral; and selects a closest match between the output signals of the sensor set and the disease-specific pattern and all contained within (d) an overall housing.

Inventive concept 18. The method of inventive concept 17, wherein the apparatus will detect a viral or bacterial disease in a subject by measuring and analysing and making comparison with the digital fingerprints of the odours or Volatile Organic Compounds released from the subject's body sweat previously absorbed into a previously worn piece of clothing; and, the system comprising: (a) a housing into which the sample of clothing can be inserted also containing a heater whose temperature can be controlled between 30 and 90 degrees Celsius, (b) an air sampling unit able to take a sample of the odours or Volatile Organic Compounds emitted by the clothing heated inside said container and to divert said sample to enter the sensor set; (b) a selected definitive sensor set comprising at least two sensors reactive to the presence of specific odours or Volatile Organic Compounds (VOCs) in the air sample taken from the clothing; and (c) a processing unit comprising a pattern recognition analyser, wherein the pattern recognition analyser receives output signals of the sensor set; compares them to disease-specific patterns derived from a database of response patterns of the sensor set exposed to the body sweat samples of subjects with known disease, wherein each of the disease-specific patterns is characteristic of a particular disease, both bacteriological or viral; and selects a closest match between the output signals of the sensor set and the disease-specific pattern and all contained within (d) an overall housing.

Inventive concept 19. The method of inventive concept 17, wherein at least one other sensor is selected from the group consisting of surface acoustic wave sensors, quartz crystal resonators, metal oxide sensors, dye-coated fibre optic sensors, micro-machined cantilever arrays, composites having regions of conducting material and regions of insulating organic material, composites having regions of conducting material and regions of conducting or semi-conducting organic material, chemically-sensitive resistor or capacitor film, semi-conducting polymer sensors, metal-oxide-semiconductor field effect transistors, and bulk organic conducting polymeric sensors.

Inventive concept 20. The method of inventive concept 17, wherein said test sample comprises the odours or Volatile Organic Compounds emanating from either breath samples released from a previously worn face mask or from body sweat samples released from previously worn clothing; or from a breath sample.

Inventive concept 21. The method of inventive concept 17, wherein the container 5 for enclosing the adsorber sample is made removable and disposable to enhance the biosecurity of the system.

Inventive concept 22. The method of inventive concept 17, wherein the data is analysed by comparing the data to a database containing data profiles from a plurality of detectable signals and identifying the at least one specific odour or Volatile Organic Compound present in the sample thereby characterizing the disease or combinations of diseases.

Inventive concept 23. The method of inventive concept 17, wherein the disease or diseases causing the illness in the subject is of bacteriological or viral origin. By comparing the results with the library of stored VOC digital fingerprints the system will differentiate between a disease of bacteriological origin and one of viral infection origin thus establishing if the illness can be treated successfully with antibiotics.

Inventive concept 24. The system according to inventive concept 17, wherein the database comprises response patterns of the sensor set when sampling odours or Volatile Organic Compounds emanating from the group consisting of breath samples and body sweat samples having a bodily origin of subjects suffering from a known disease.

Inventive concept 25. The system according to inventive concept 17, with a method of diagnosing, screening or monitoring the presence of a disease or diseases in a test subject in a non-invasive manner by sampling the atmosphere around the subject, the method comprising the steps of: (a) providing a system according to claim 1; (b) exposing the sensor set to the odours or Volatile Organic Compounds emanating from the group consisting of breath samples or body sweat samples; (c) measuring the output signals of the sensor set upon exposure to the said sample; (d) comparing the output signals using a pattern recognition analyser to the database—derived disease-specific patterns; and (e) selecting the closest match between the output signals of the sensor set and the database-derived disease specific patterns and applying a tolerance between the output signals of the sensor set and the database-derived disease specific patterns, to indicate a Pass or a Fail (Infected or Non-Infected) condition.

Inventive concept 26. The system according to inventive concept 17, with the addition of a wireless communication device, including but not limited to Wi-Fi; Bluetooth® and Telephone SMS messaging, to enable a warning signal to be transmitted in the event of a disease or diseases being detected in the location in which it is being monitored.

Inventive concept 27. The system according to inventive concept 17, with the addition of an air filter unit, including but not limited to a capsule of carbon granules or carbon impregnated cloth, and being capable of adsorbing odours and Volatile Compounds, said air filter unit purifying the air used to clean and purge the sensor set before the next sampling and measurement sequence.

Inventive concept 28. The system according to inventive concept 17, with the addition of an air filter unit, including but not limited to a capsule of adsorbent material carbon granules or carbon impregnated cloth, said air filter unit being inserted ahead of the air outlet to limit the risk of releasing any infected material into the general atmosphere.

Inventive concept 29. The system according to inventive concept 17, with the provision of a computer or other memory and being capable of recording every measurement taken by every sensor in the sensor set together with date and time records; these records being downloaded through an electrical or wireless connection to said apparatus.

Inventive concept 30. The method of inventive concept 17, wherein the disease-specific patterns in the database can be added to with new disease-specific patterns characterising a disease or diseases not previously included in the database of response patterns of the sensor set to the odour or Volatile Organic Compound emissions from the breath or body sweat of subjects with known diseases.

Inventive concept 31. In the method of inventive concept 17, wherein the cases of diseases caused by viral infections the indicative combinations of odours or Volatile Organic Compounds will include, but not be limited to, chemical compounds from the aldehydes, ketones and alcohols group and in the case of diseases caused by bacterial infections the indicative combinations of odours or Volatile Organic Compounds will include, but not be limited to, chemical compounds from the alcohols, aldehydes and sulphides group.

Inventive concept 32. The system according to inventive concept 17, with the sensitivity of the system being enhanced by the insertion of a piece of material, known to absorb odours or Volatile Organic Compounds, into the face mask or clothing. Suitable commercially available absorbent materials include but are not limited to, Tenax TA, Tenax GR, Carbotrap, Carbopack B and C. Carbotrap C, Carboxen, Carbosieve SIlI, Proapak, Spherocarb, and combinations thereof; preferred adsorbent combinations include, but are not limited to, Tenax GR and Carbopack B: Carbopack B and Carbosieve SIII; and Carbopack C and Carbopack Band Carbosieve SIII or Carboxen 1000. Those skilled in the art will know of other suitable absorbent materials.

Inventive concept 33. The method of inventive concept 17, where the breath sample is collected in a disposable device comprising a tube which is inserted into a housing carrying a panel of adsorbent material. The tube is inserted into the subject's mouth and then the subject breaths out and the VOCs in the subject's breath are collected in the adsorbent material which covers the housing. After an appropriate number of breaths; dependent upon the VOC types to be collected; the tube is removed and the housing & adsorber is taken to the measuring and analysing apparatus. The adsorber material, is one known to absorb odours or Volatile Organic Compounds. Suitable commercially available absorbent materials include but are not limited to, Tenax TA, Tenax GR, Carbotrap, Carbopack B and C. Carbotrap C, Carboxen, Carbosieve SIII, Proapak, Spherocarb, and combinations thereof; preferred adsorbent combinations include, but are not limited to, Tenax GR and Carbopack B: Carbopack B and Carbosieve SIII; and Carbopack C and Carbopack Band Carbosieve SIII or Carboxen 1000. Those skilled in the art will know of other suitable absorbent materials.

Inventive concept 34. The adsorbed sample of odours or VOCs can be measured and analysed in the apparatus immediately after it is collected or it can be stored in a sealed container for subsequent measurement and analysis. The adsorbed sample can be stored for periods exceeding one year if it is held at a temperature of below 10 degrees Celsius.

1. Apparatus for detecting the presence in an enclosed environment of a subject or subjects infected with viral, bacterial and/or parasitic disease or diseases, the apparatus comprising:
(a) an air sampling unit able to take an air sample of the atmosphere in the enclosed environment and to divert said sample for sensing;
(b) a selected definitive sensor set comprising at least two sensors reactive to the presence of specific odours or Volatile Organic Compounds (VOCs) in the air sample taken from the environment;
(c) a processing unit comprising a pattern recognition analyser, wherein the pattern recognition analyser receives output signals of the sensor set, compares them to disease-specific patterns derived from a database of response patterns of the sensor set exposed to the totality of the bodily emissions of subjects with known disease or diseases, wherein each of the disease-specific patterns is characteristic of a particular disease, selected from bacteriological, viral and parasitic disease, and selects a closest match between the output signals of the sensor set and the disease-specific pattern; and
(d) a control system that triggers the sampling of the air space of the environment at pre-determined times or intervals for rendering the apparatus entirely automatic and self-contained in operation,
wherein the air sampling unit comprises: a surface for capturing VOCs from the air sample; and a heater for heating the surface to release captured VOCs when diverting the air sample for sensing.

2. The apparatus of claim 1, wherein at least one sensor is selected from the group consisting of surface acoustic wave sensors, quartz crystal resonators, metal oxide sensors, dye-coated fibre optic sensors, micro-machined cantilever arrays, composites having regions of conducting material and regions of insulating organic material, composites having regions of conducting material and regions of conducting or semi-conducting organic material, chemically-sensitive resistor or capacitor film, semi-conducting polymer sensors, metal-oxide-semiconductor field effect transistors, and bulk organic conducting polymeric sensors.

3. The apparatus of claim 1, wherein said test sample is configured to comprise the ambient atmosphere of the enclosed environment together with the totality or combined total of the odours or Volatile Organic Compounds (VOCs) selected from the group consisting of breath samples, sweat samples, urine samples, vaginal samples, faeces samples, tissue samples; eructation samples, flatulence samples and any other samples having a bodily origin.

4. The apparatus of claim 1, wherein the processing unit is configured to analyse data by comparing the data to a database containing data profiles from a plurality of detectable signals and identifying the at least one specific odour or Volatile Organic Compound present in the sample thereby characterizing the disease or combinations of diseases.

5. The apparatus of claim 1, wherein the air sampling unit is configured to pass air of the enclosed environment over the surface.

6. The apparatus according to claim 5, wherein the air sampling unit is configured to pass a volume of air of the enclosed environment over the surface which is greater than the volume of the air sample diverted for sensing.

7. The apparatus according to claim 6, wherein the air sampling unit is configured to pass a volume of air of the enclosed environment over the surface which is at least 100 times greater than the volume of the air sample diverted for sensing.

8. The apparatus of claim 1, wherein the surface for capturing VOCs from the air sample is configured to adsorb or absorb VOCs from the air sample.

9. The apparatus according to claim 8, wherein the surface for capturing VOCs from the air sample is part of a material selected from the group consisting of a nano-porous material, a microporous material, a chemically reactive material, a nonporous material and combinations thereof.

10. The apparatus of claim 1, wherein at least one of the at least two sensors in the sensor set has a sensitivity of at least 5 parts per billion by volume.

11. The apparatus of claim 1, wherein the air sampling unit is configured to heat the heater to a temperature to release a specific VOC captured by the surface.

12. The apparatus of claim 1, further comprising an air filter unit configured to remove Volatile Organic Compounds from ambient air and provide purified air for cleaning and purging the sensor set before sensing the air sample.

13. A method of diagnosing, screening or monitoring the presence of a viral, bacterial and/or parasitic disease or diseases in a test subject in a non-invasive manner by sampling the atmosphere around the subject in an enclosed environment, the method comprising the steps of:
(a) providing apparatus comprising: (i) an air sampling unit able to take an air sample of the atmosphere in the enclosed environment and to divert said sample for sensing, the air sampling unit comprising a surface for capturing Volatile Organic Compounds (VOCs) from the air sample; (ii) a selected definitive sensor set comprising at least two sensors reactive to the presence of specific odours or VOCs in the air sample taken from the environment; and (iii) a database of response patterns of the sensor set exposed to a totality of bodily emissions of subjects with known disease or diseases, wherein each of the disease-specific patterns is characteristic of a particular disease, selected from bacteriological, viral and parasitic disease;
(b) capturing on the surface of the air sampling unit VOCs from an air sample, the VOCs being selected from the group consisting of breath samples, urine samples, vaginal samples, faeces samples, tissue samples, eructation samples, flatulence samples and any other samples having a bodily origin of the test subject;
(c) heating the surface to release captured VOCs and diverting the released VOCs to the sensor set;
(d) measuring the output signals of the sensor set upon exposure to the said sample;
(e) comparing the output signals using a pattern recognition analyser to the database-derived disease-specific patterns; and
(e) selecting the closest match between the output signals of the sensor set and the database-derived disease-specific patterns.

14. The method of claim 13, further comprising applying a tolerance between the output signals of the sensor set and the database-derived disease-specific patterns, and indicating a Pass or a Fail (Infected or Non-Infected) condition in dependence upon whether the tolerance is exceeded or not.

15. The method according to claim 13, further comprising the step of passing air from the atmosphere around the subject over the surface to concentrate the Volatile Organic Compounds captured on the surface.

16. The method according to claim 15, wherein passing air from the atmosphere around the subject over the surface to concentrate the Volatile Organic Compounds captured on the surface by a factor of at least 10.

17. The method according to claim 13, wherein the enclosed environment is selected from the group consisting of: an aircraft cabin; a ship or boat cabin; a motor vehicle interior; an office; a home; a school; a health or medical facility; a sports or training facility a poultry house; a cattle barn; and a pig (swine) pen.

18. The method of claim 13, further comprising adding a disease-specific pattern characterising a disease or diseases not previously included in the database of response patterns of the sensor set to the bodily emissions of subjects with known diseases.

19. The method of claim 13, wherein the Volatile Organic Compounds of interest when monitoring for a disease caused by a viral infection include chemical compounds selected from the aldehydes, ketones and alcohols groups, and the Volatile Organic Compounds of interest when monitoring for a disease caused by a bacterial infection include chemical compounds from the alcohols, aldehydes and sulphides groups.

20-36. (canceled)
